(19) United States
(12) Patent Application Publication
(10) Pub. No.: US 2019/0062988 A1
(43) Pub. Date: Feb. 28, 2019

(54) SACHET FOR WASHING COSMETIC SPONGES

(71) Applicant: Joanne Duncan-Carnesciali, New York, NY (US)

(72) Inventor: Joanne Duncan-Carnesciali, New York, NY (US)

(21) Appl. No.: 15/692,782

(22) Filed: Aug. 31, 2017

(51) Int. Cl. D06F 95/00; B65D 33/24; B08B 3/04; A61K 8/02

(52) U.S. Cl. CPC D06F 95/006; B65D 33/24; A47K 7/02; A61K 8/0208; B08B 3/04

(57) ABSTRACT

A sachet used for packaging and washing cosmetic sponges. The sachet is made of a permeable material so water may pass freely into and out of the sachet. The sachet includes an opening forming an entrance into the sachet. The sachet further includes a connector operable to open and close the opening and thereby open and close the entrance.

### BACKGROUND OF THE INVENTION

The present invention relates to packaging and cleaning cosmetic sponges and, more particularly, to an environmentally sustainable sachet used for washing cosmetic sponges within a washing machine.

Cosmetics are substances or products used to enhance or alter the appearance of the body. Many cosmetics are designed for use of applying to the face, body and hair. Cosmetics that are meant to be used on the face and eye area are usually applied with a brush, a cosmetic sponge, or the fingertips. Cosmetic sponges must be washed after use. Currently, cosmetic sponges are individually hand washed, which is time consuming and requires excessive use of water, time and cleaning solution.

As can be seen, there is a need for an environmentally sustainable method and apparatus for effectively and efficiently washing of cosmetic sponges.

### SUMMARY OF THE INVENTION

In one aspect of the present invention, a method of cleaning cosmetic sponges, the method comprises the steps of: providing a sachet made of a permeable material, the sachet comprising a connector operable to open and close an opening forming an entrance into the sachet; opening the entrance of the sachet via the connector; placing a plurality of used cosmetic sponges through the entrance and into the sachet; closing the entrance of the sachet via the connector; placing the sachet into a washing machine; and powering the washing machine.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention shown in use;

FIG. 2 is a perspective view of an embodiment of the present invention in an opened position;

FIG. 3 is a section view of an embodiment of the present invention being washed; and

FIG. 4 is a perspective view of an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a sachet used for packaging and washing cosmetic sponges. The sachet for cosmetic sponges solves the problem of plastic waste and pollution and the frequent and time-consuming manual washing of individual cosmetic sponges. The sachet allows the user to wash cosmetic sponges in the same manner as one washes ordinary laundry, reducing water waste, cost, time, effort and messiness. The present invention further provides a means for the user to store and carry the cosmetic in a sanitary manner when travelling.

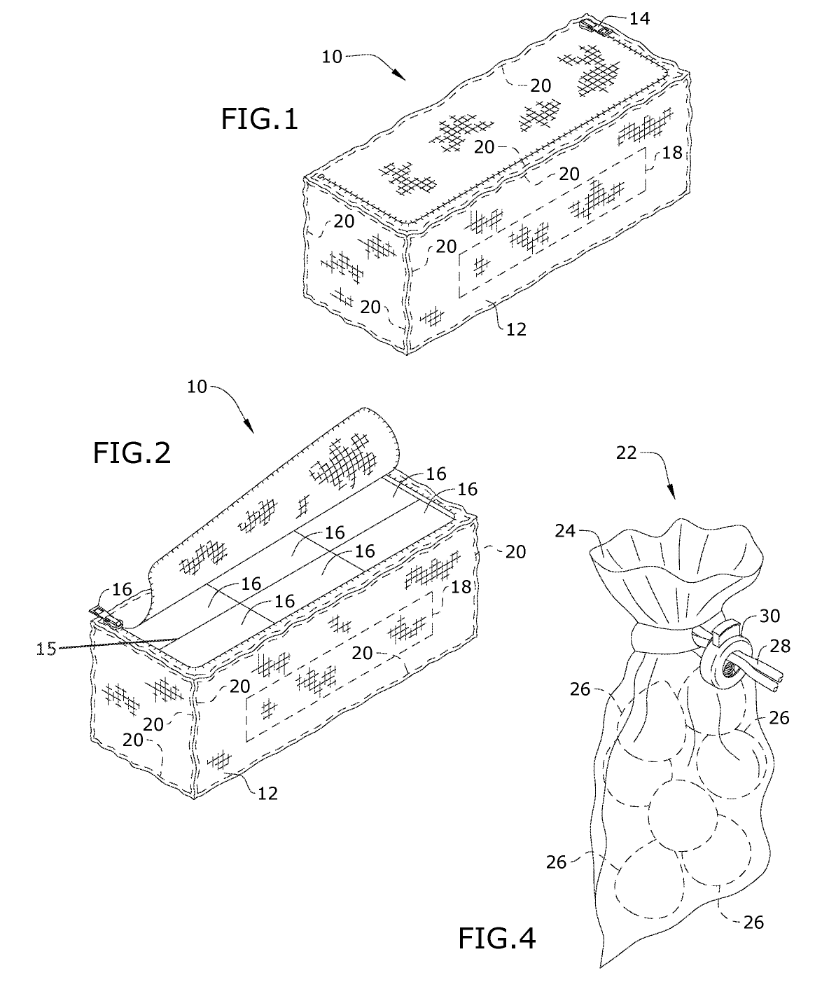

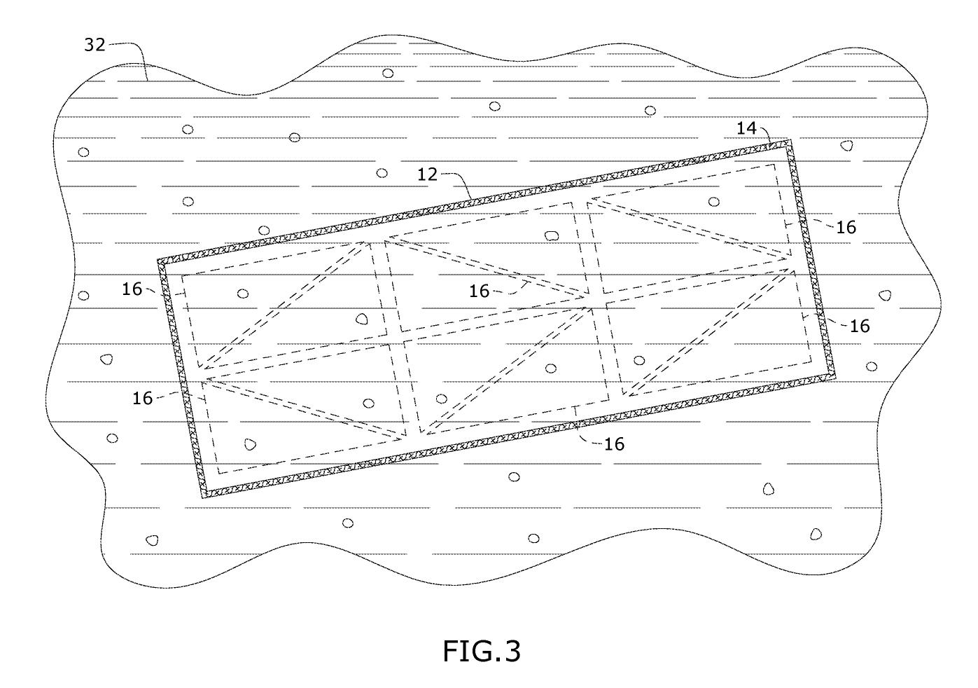

Referring to FIGS. 1 through 3, the present invention includes a sachet 10 used for packaging, washing and storing cosmetic sponges 16. The sachet 10 is made of a permeable material 12 so soapy water 32 may pass freely into and out of the sachet 10. The sachet 10 includes an opening forming an entrance into the sachet 10. The sachet 10 further includes a connector 14 operable to open and close the opening and thereby open and close the entrance.

The permeable material forming the sachet may include a durable soft machine washable utility fabric. For example, the sachet 10 may be made of a woven fabric, a mesh material and the like. The woven fabric may be connected together by stitching 20. In certain embodiments, a logo may be stitched to or printed on a logo area 18 of an outer surface of the sachet 10.

The sachet 10 may include a rectangular shape. In such embodiments, the connector 14 may be a zipper. The zipper may be used to open and close the entrance. A user may open the sachet 10 using the zipper, place the cosmetic sponges 16 into the sachet 10 and then close the sachet using the zipper. The sachet may be placed into a washing machine and may be washed in soapy water 32. The sponges 16 may include an incline plane forming a ramp shape or other shape. The ramp shaped sponges 16 may stack against one another when packaged within the sachet 10. The rectangular shaped sachet 10 may be used as an alternative to plastic packaging unit for manufacturers and venders of cosmetic sponges in greater quantities.

The washing machine may be a standard washing machine powered by electricity. The washing machine may use water and detergent to wash and clean the cosmetic sponges 16 within the sachet 10.

In certain embodiments, the sachet 10 includes dividers 15. The dividers 15 are disposed within the sachet 10 and form a plurality of compartments within the sachet 10. Each compartment may fit a single cosmetic sponge 16 within.

As illustrated in the FIG. 4, a sachet 22 may include a bag shape. The sachet 22 may be made of a permeable material 24. The bag shaped sachet also includes a connector 28, 30. The connector 28, 30 may include an elastic cord 28 and a cord stop 30. The elastic cord 28 is loosened using the cord stop 30 to open the entrance of the sachet 22. A plurality of cosmetic sponges 26 may be placed through the entrance and within the sachet 22. The entrance of the sachet 22 is closed by tightening the elastic cord 28 using the cord stop 30. The sachet 22 is then placed in a washing machine to be washed.

A method of cleaning cosmetic sponges may include the following steps: opening the entrance of the sachet via the connector; placing a plurality of used cosmetic sponges through the entrance and into the sachet; closing the entrance of the sachet via the connector; pretreating the exterior of the sachet and sponges with a cleaner; placing the sachet into a washing machine; adding detergent to the washing machine; and powering the washing machine. The plurality of used cosmetic sponges is a plurality of sponges each having a layer of makeup disposed on a surface.

It should be understood, of course, that the foregoing relates to exemplary embodiments of the invention and that modifications may be made without departing from the spirit and scope of the invention as set forth in the following claims.

1. A method of cleaning cosmetic sponges, the method comprising the steps of:
providing a sachet made of a permeable material, the sachet comprising a connector operable to open and close an opening forming an entrance into the sachet;
opening the entrance of the sachet via the connector;
placing a plurality of used cosmetic sponges through the entrance and into the sachet;
closing the entrance of the sachet via the connector;
placing the sachet into a washing machine; and
powering the washing machine.

2. The method of claim 1, further comprising the step of adding detergent to the washing machine prior to powering the washing machine.

3. The method of claim 1, wherein the sachet comprises a rectangular shape.

4. The method of claim 3, wherein the connector is a zipper.

5. The method of claim 1, wherein the sachet comprises a bag shape.

6. The method of claim 5, wherein the connector is an elastic cord and a cord stop secured to the elastic cord.

7. The method of claim 1, wherein the permeable material is a woven fabric.

8. The method of claim 1, wherein the permeable material is a mesh material.

9. The method of claim 1, wherein plurality of used cosmetic sponges comprises a plurality of sponges each having a layer of makeup disposed on a surface.

10. The method of claim 1, further comprising dividers disposed within the satchel, wherein the dividers form a plurality of compartments each sized to fit one of the plurality of sponges within.

11. A method of packaging cosmetic sponges comprising the steps of:
providing a sachet made of a mesh or woven fabric, the sachet comprising a connector operable to open and close an opening forming an entrance into the sachet;
opening the entrance of the sachet via the connector;
placing a plurality of cosmetic sponges through the entrance and into the sachet; and
closing the entrance of the sachet via the connector.

12. An apparatus comprising:
a plurality of cosmetic sponges; and
a sachet made of a permeable material, the sachet comprising a connector operable to open and close an opening forming an entrance into the sachet, wherein
the plurality of cosmetic sponges are packaged within the sachet.

13. The apparatus of claim 12, wherein the sachet comprises a rectangular shape and each of the plurality of cosmetic sponges comprise an incline plane forming a ramp shape.
